(19) United States
(12) Patent Application Publication
(10) Pub. No.: US 2017/0188810 A1
(43) Pub. Date: Jul. 6, 2017

(54) AUTOMATIC EYESIGHT TEST DEVICE AND AUTOMATIC EYESIGHT TEST METHOD

(71) Applicant: VIEWMTECHNOLOGY CO., LTD., Seoul (KR)

(72) Inventors: Ki Gon KIM, Seoul (KR); Kwang Kyu LEE, Seoul (KR)

(73) Assignee: VIEWMTECHNOLOGY CO., LTD., Seoul (KR)

(21) Appl. No.: 15/315,383

(22) PCT Filed: Dec. 9, 2014
(86) PCT No.: PCT/KR2014/012040

§ 371 Date: Dec. 1, 2016

(30) Foreign Application Priority Data
Jun. 12, 2014 (KR) 10-2014-0071367

(51) Int. Cl. A61B 3/032; A61B 3/00

(52) U.S. Cl. CPC A61B 3/032; A61B 3/0041; A61B 3/0033

(57) ABSTRACT

Provided is an automatic eyesight test device comprising: a display unit for displaying an eyesight test chart image including a first figure expressed in a line and a second figure expressed in a line in the interior of the first figure; and an input unit for receiving a user input having responded to the displayed eyesight test chart image, wherein the interval between a first point of a line expressing the first figure and a point corresponding to a line expressing the second figure is narrower than the interval between a second point of the line expressing the first figure and the point corresponding to the line expressing the second figure.

### TECHNICAL FIELD

The present invention relates to an automatic eyesight test device and an automatic eyesight test method and, more particularly, to an automatic eyesight test device and automatic eyesight test method, which enable an accurate eyesight test through simple user input using a new eyesight test chart different from an existing eyesight test chart.

### BACKGROUND ART

An eyesight test is performed in an ophthalmic clinic, an optician's shop and a health medical examination. In general, an eyesight test is performed by an optician or a nurse who directly checks the eyes of a testee in the state in which a light for an eyesight test chart is 200 lux in brightness in a room having a uniform light and an eyesight test chart has been installed in the eye height of a testee. The testee at a specific distance (5 m) sees the eyesight test chart at the front, and the eyesight of the right eye of the person is measured with the left eye of the person covered. The left eye of the person is measured using the same method. A corresponding number read by the person from a large eyesight chart to a gradually smaller eyesight chart in a minimum eyesight chart (or index) becomes the eyesight of the person.

In an existing eyesight test, in order to check the eyesight of a testee, basically, the person has to be guided by an optician or a nurse and notifies the optician, etc. of a response to an eyesight chart indicated by the optician or the nurse, so the eyesight test is performed.

The existing eyesight test method causes several problems. First, manpower for an eyesight test is required, the accuracy of an eyesight test is different depending on the skill level of a person for an eyesight test, and there is a variation in the eyesight test.

An existing eyesight test chart may include Landolt ring or Snellen's eyesight chart. Landolt ring or Snellen's eyesight chart requires a minimum operation of a testee or requires an intellectual capacity. For example, if the Landolt ring eyesight chart is used, an accurate response from an infant or the aged who has a low intellectual capacity cannot be obtained because Landolt ring is opened at direction of 360 degrees. Accordingly, accuracy is not guaranteed. For this reason, a separate eyesight test chart must be provided for infants, etc.

A known automation device for an eyesight test may be used. If the automation device is used, it is preferred that user input has a simple configuration. If user input is complicated, it is impossible for such an automation device to be used by a testee. Furthermore, if an automation device is used, it may be preferred that a short-distance or long-distance eyesight test method is provided.

Accordingly, there is a need for an automatic eyesight test device and an automatic eyesight test method capable of solving various problems caused in the existing eyesight test method.

### DISCLOSURE OF INVENTION

### Technical Problem

The present invention has been made to solve the aforementioned problems, and an object of the present invention is to provide an automatic eyesight test device and automatic eyesight test method, which can perform a unified eyesight test regardless of the type of a testee, such as children, the old, the infirm, etc.

Furthermore, an object of the present invention is to provide an automatic eyesight test device and automatic eyesight test method, which enable an eyesight test to be automatically performed in response to simple user input.

Furthermore, an object of the present invention is to provide an automatic eyesight test device and automatic eyesight test method, which enable a long-distance eyesight test and a short-distance eyesight test to be performed at the same time.

Furthermore, an object of the present invention is to provide an automatic eyesight test device and automatic eyesight test method, which can perform an eyesight test efficiently through simple user input using a restricted eyesight chart in a restricted input/output environment of an automation device.

Furthermore, an object of the present invention is to provide an automatic eyesight test device and automatic eyesight test method, which can reduce a measurement variation in a testee and reduce personal expenses and a test time for an eyesight test.

Technical objects to be achieved by the present invention are not limited to the aforementioned object, and those skilled in the art to which the present invention pertains may evidently understand other technical objects from the following description.

### Technical Solution

An automatic eyesight test device for achieving the above objects includes a display unit for displaying an eyesight test chart image including a first figure expressed in a line and a second figure expressed in a line within the line of the first figure and an input unit for receiving user input which has responded to the displayed eyesight test chart image. An interval between a first point of the line expressing the first figure and a corresponding point of the line expressing the second figure is narrower than an interval between a second point of the line expressing the first figure and a corresponding point of the line expressing the second figure.

Furthermore, the automatic eyesight test device further includes a control unit for generating the eyesight test chart image comprising a first figure and a second figure. The control unit generates a subsequent eyesight test chart image which includes a first figure expressed in a line and a second figure expressed in a line within the line of the first figure and differs from a previously generated eyesight test chart image in response to user input received through the input unit, and outputs the generated subsequent eyesight test chart image to the display unit. The size of a first figure and second figure included in the previously generated eyesight test chart image and the size of the first figure and second figure included in the subsequent eyesight test chart image are different. The size of the first figure and second figure included in the subsequent eyesight test chart image is determined by the user input.

Furthermore, the previously generated eyesight test chart image corresponds to a first eyesight value, and the subsequent eyesight test chart image corresponds to a second eyesight value different from the first eyesight value.

Furthermore, the automatic eyesight test device further comprises an optical unit including a plurality of lenses and an output unit outputting eyesight test results. The control unit performs an eyesight test of long-distance mode or short-distance mode while operating in conjunction with user input using the plurality of lenses of the optical unit and outputs eyesight test results to the output unit.

Furthermore, the first figure and the second figure are figures of a ring type, the control unit outputs only the subsequent eyesight test chart image to the display unit in response to the user input, and the user input is a response to a form of an eyesight test chart image visually recognized by a user with respect to a single eyesight test chart image corresponding to the first eyesight value.

Furthermore, an automatic eyesight test method for achieving the above object comprises the steps of (a) displaying an eyesight test chart image comprising a first figure expressed in a line and a second figure expressed in a line within the line of the first figure and (b) receiving user input which has responded to the displayed eyesight test chart image. An interval between a first point of the line expressing the first figure and a corresponding point of the line expressing the second figure is narrower than an interval between a second point of the line expressing the first figure and a corresponding point of the line expressing the second figure.

Furthermore, the automatic eyesight test method further comprises the step of (c) displaying an eyesight test chart image including a first figure expressed in a line and a second figure expressed in a line within the line of the first figure in response to the user input. The size of the first figure and second figure included in the eyesight test chart image at the step (a) and the size of the first figure and second figure included in the eyesight test chart image at the step (c) are different. The size of the first figure and second figure at the step (c) is determined by user input.

Furthermore, the eyesight test chart image at the step (a) corresponds to a first eyesight value, and the eyesight test chart image at the step (c) corresponds to a second eyesight value different from the first eyesight value.

Furthermore, the user input is a response to the eyesight test chart image visually recognized by a user with respect to the first point of the line expressing the first figure and the corresponding point of the line of the second figure.

Furthermore, the user input is input indicating whether the first figure and the second figure have visually overlapped with respect to the first point and the corresponding point or input indicative of a direction recognized by the user for the first point and the corresponding point.

Furthermore, the first figure and the second figure are figures of a ring type, the step (a) comprises displaying only a single eyesight test chart image corresponding to the first eyesight value, the step (c) comprises displaying only a single eyesight test chart image corresponding to the second eyesight value, and the step (b) comprises receiving a response to a form of an eyesight test chart image visually recognized by a user with respect to a single eyesight test chart image corresponding to the first eyesight value as the user input.

### Advantageous Effects

An automatic eyesight test device and automatic eyesight test method according to an embodiment of the present invention, such as those described above, have an advantage in that a unified eyesight test can be performed regardless of the type of a testee, such as children, the old, the infirm, etc.

Furthermore, an automatic eyesight test device and automatic eyesight test method according to an embodiment of the present invention, such as those described above, have an advantage in that an eyesight test can be automatically performed in response to simple user input.

Furthermore, an automatic eyesight test device and automatic eyesight test method according to an embodiment of the present invention, such as those described above, have an advantage in that a long-distance eyesight test and a short-distance eyesight test can be performed at the same time.

Furthermore, an automatic eyesight test device and automatic eyesight test method according to an embodiment of the present invention, such as those described above, have an advantage in that an eyesight test can be performed efficiently through simple user input using a restricted eyesight chart in a restricted input/output environment of an automation device.

Furthermore, an automatic eyesight test device and automatic eyesight test method according to an embodiment of the present invention, such as those described above, have advantages in that a measurement variation in a testee can be reduced and personal expenses and a test time for an eyesight test can be reduced.

Advantages which may be obtained in the present invention are not limited to the aforementioned advantages, and various other advantages may be evidently understood by those skilled in the art to which the present invention pertains from the following description.

### BRIEF DESCRIPTION OF DRAWINGS

FIG. 1 is a diagram showing an exemplary external appearance of an automatic eyesight test device.

FIG. 2 is a diagram showing an exemplary block diagram of the automatic eyesight test device.

FIG. 3 is a diagram showing exemplary eyesight test chart images which are used in the present invention and displayed on a display unit.

FIG. 4 is a diagram showing an exemplary internal configuration of the automatic eyesight test device for an eyesight test in long/short-distance mode.

FIG. 5 is a diagram showing a flowchart for an eyesight test.

FIG. 6 is a diagram showing a flowchart which uses the eyesight chart according to the present invention and is used to test binocular, left-eye or right-eye eyesight through simple user input.

<Description of reference numerals>

| | |
|---|---|
| 100: automatic eyesight test device | |
| 101: input unit | 103: storage unit |
| 105: external interface unit | 107: output unit |
| 109: sensing unit | 111: display unit |
| 113: lighting unit | 115: optical unit |
| 117: control unit | 119: connection unit |

### MODE FOR CARRYING OUT THE INVENTION

The above objects, characteristics, and merits will become more apparent from the following detailed description taken in conjunction with the accompanying drawings, and thus those skilled in the art to which the present invention pertains may readily implement the technical spirit of the present invention. Furthermore, in describing the present invention, a detailed description of a known art related to the present invention will be omitted if it is deemed to make the gist of the present invention unnecessarily vague. Preferred embodiments in accordance with the present invention are described in detail below with reference to the accompanying drawings.

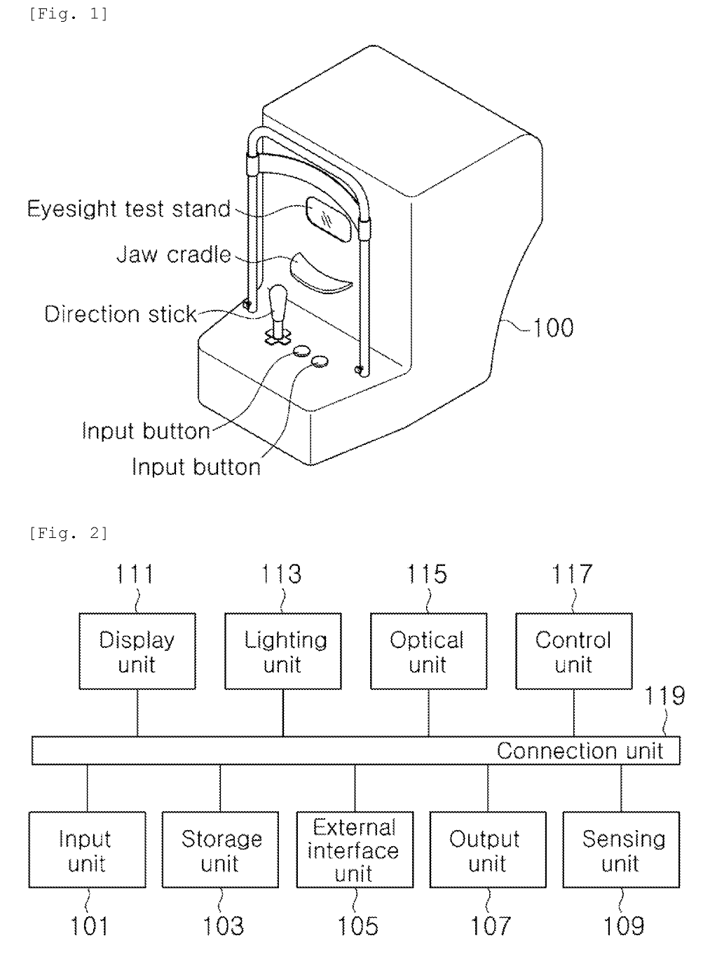

FIG. 1 is a diagram showing an exemplary external appearance of an automatic eyesight test device 100.

Referring to FIG. 1, the automatic eyesight test device 100 includes a jaw cradle and an eyesight test stand and includes a direction stick and one or more buttons. The direction stick or a specific one button may be omitted according to a modified example.

An external appearance of the automatic eyesight test device 100 is described in brief. The jaw cradle is an instrument in which the jaw of a face is safely received upon eyesight test. The eyesight test stand is configured to accommodate both eyes. An image exposed to one eye (also called a “monocular”) or both eyes (also called a “binocular”) is displayed through the eyesight test stand.

The direction stick is configured to indicate a specific direction of an image displayed through the eyesight test stand. For example, the direction stick outputs a signal indicative of a left, right, up or down direction to the inside of the automatic eyesight test device 100.

The buttons are used to input a response from a testee for an image displayed like the direction stick. Specifically, the buttons are used to display a form of figures of images that are visually recognized by a testee on the automatic eyesight test device 100. For example, one button is used for a testee to indicate whether the figures of images have overlapped. The other button is used for a testee to indicate the figures of images have been separated (not overlapped). Two or more buttons do not need to be essentially used, and a recognized form of a figure displayed in an image may be indicated through a single button.

For an eyesight test, only the button is used or only the direction stick is used or both may be used. The button or the direction stick may be omitted depending on a using pattern of an eyesight test. The button is a push type button or a touch type button.

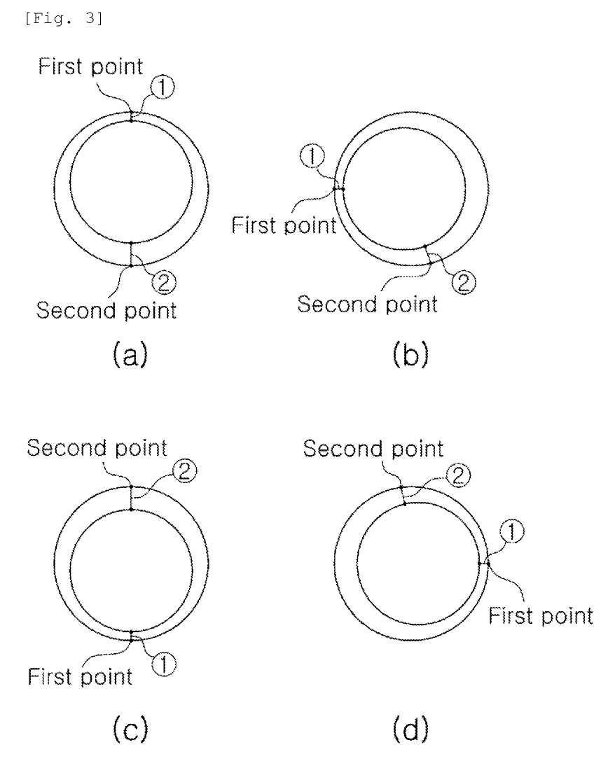

The automatic eyesight test device 100 is described in more detail with reference to FIGS. 2 to 6.

FIG. 2 is a diagram showing an exemplary block diagram of the automatic eyesight test device 100.

Referring to FIG. 2, the automatic eyesight test device 100 includes an input unit 101, a storage unit 103, an external interface unit 105, an output unit 107, a sensing unit 109, a display unit 111, a lighting unit 113, an optical unit 115, a control unit 117 and a connection unit 119. Some of the blocks included in FIG. 2 may be omitted depending on a modified example, and another block not included in the block diagram may be further included in the block diagram.

Each of the blocks of the automatic eyesight test device 100 is described. The input unit 101 receives user input from a testee. The input unit 101 includes the direction stick and the one or more buttons, receives input by a testee from the direction stick and/or the button, and outputs the input to the control unit 117. The user input received through the input unit 101 is used to indicate a form of an image, recognized by the testee, in response to an image displayed through the eyesight test stand.

Furthermore, the input unit 101 may receive input indicative of setting data by an optician or nurse who performs an eyesight test through the automatic eyesight test device 100. For example, the input unit 101 may include a setting switch in addition to the button or direction stick used by a testee so that long-distance mode (e.g., 5 m) or short-distance mode (e.g., 50 cm) used for an eyesight test is set depending on a setting value of the setting switch.

The storage unit 103 stores various types of data and programs, etc. The storage unit 103 includes volatile memory, nonvolatile memory and/or high-capacity storage medium such as hard disk, etc. For example, the storage unit 103 includes the data of an eyesight test chart according to an embodiment of the present invention, which is used in the automatic eyesight test device 100, a test program performing eyesight test using the eyesight test chart data and results data of eyesight test.

The data indicative of the eyesight test chart constitutes eyesight values and corresponding eyesight test chart images. The number of eyesight test chart images corresponding to an eyesight value may be one or plural. The data of the eyesight test chart includes a plurality of eyesight values and corresponding eyesight test chart images, and may enable to test eyesight of 7-step, 9-step or 13-step depending on the number of eyesight test chart images. Eyesight test chart images corresponding to each step preferably include two figures, and the size of a figure of images of a specific one step is different from the size of a figure of images of another step. The automatic eyesight test device 100 performs an eyesight test in response to user input depending on a form of a figure recognized by a testee, and the results of the eyesight test are output. Displayed shapes of figures of images are descried with reference to FIG. 3.

In this case, an eyesight test chart image stored in the storage unit 103 does not need to be essentially expressed or stored in the form of color data (e.g., RGB, YUV or black and white data). An eyesight test chart image stored in the storage unit 103 is enough to be data capable of generating data to be output through the display unit 111. Accordingly, the eyesight test chart image includes color data to be output to the display unit 111 or includes data capable of generating color data.

The external interface unit 105 is an interface for operating in conjunction with an external device of the automatic eyesight test device 100. The external interface unit 105 may perform short-distance communication for operating in conjunction with a remote controller, a fax, a personal computer (PC), etc. The external interface unit 105 includes an interface for an infrared remote controller signal, Ethernet, Bluetooth or a communication interface for fax, and transfers eyesight test results or receives control data under the control of the control unit 117, so the control unit 117 may perform an operation according to the control data.

The output unit 107 outputs eyesight test results determined by the control unit 117. The output unit 107 includes a display module such as a Liquid Crystal Display (LCD) and a Light Emitting Diode (LED), a printer module which may be embedded in the case (instrument) of the automatic eyesight test device 100 and/or an interface for interfacing with an external printer.

The output unit 107 may further include a speaker. The speaker may output a voice indicative of eyesight test results and may also output a guide comment for guiding a testee to an eyesight test under the control of the control unit 117.

The sensing unit 109 recognizes a testee for an eyesight test. An eyesight test may be started by the control unit 117 in response to the recognition of a testee by the sensing unit 109. The sensing unit 109 may include a pressure sensor for detecting pressure applied to the jaw cradle, a temperature sensor for detecting a body temperature of a testee sensed in the eyesight test stand, an infrared sensor capable of checking a testee, a circuit for outputting a signal that varies in response to pressure applied to the jaw cradle and so on.

A signal sensed by the sensing unit 109 is output to the control unit 117, and the control unit 117 may start an eyesight test.

The display unit 111 displays an image. The display unit 111 includes one or more display modules. Preferably, the display unit 111 includes two display modules. One display module (hereinafter also referred to as a “left-eye display module”) displays an image for checking the eyesight of an eye on the left (hereinafter also referred to as a “left eye”), and the other display module (hereinafter also referred to as a “right-eye display module”) displays an image for checking the eyesight of an eye on the right (hereinafter also referred to as a “right eye”).

In order to check left-eye eyesight, a corresponding left-eye display module is used. In order to check right-eye eyesight, a corresponding right-eye display module is used. In order to check binocular eyesight, the two display modules are used.

The display module may be an LCD module or an LED module. The display module receives image data from the control unit 117 through an agreed interface and displays the received image data.

Images displayed on the display unit 111 are described below.

FIG. 3 is a diagram showing exemplary eyesight test chart images which are used in the present invention and displayed on the display unit 111. The eyesight test chart images have been stored in the storage unit 103. Each eyesight chart corresponds to a specific eyesight value. FIG. 3 shows four images corresponding to a specific eyesight value. One of the four images is displayed through a display module of the display unit 111.

As may be seen from FIG. 3, an eyesight test chart image includes two figures. The two figures are figures having the same form. The two figures are rectangle, triangle, ellipse or ring (circle) type figures, for example. Each of the figures is expressed in a line. An image including the two figures is displayed on the display unit 111.

As may be seen from FIG. 3, one of the two figures has a larger size than the other figure, and one (hereinafter also referred to as an “outer figure”) of the figures includes the other (hereinafter also referred to as an “inner figure”) of the figures within its line. As may be seen from FIG. 3, the interval between one point of a line indicative of the outer figure and a corresponding point of a line indicative of the inner figure has a size (length) designated based on a corresponding eyesight value, and is different for each point.

For example, as may be seen from FIGS. 3(a) to 3(d), the interval between corresponding points of an internal figure at a specific point is the narrowest (refer to \textcircled{1} of FIGS. 3(a) to 3(d) and hereinafter also referred to as a “first point”), and the interval between corresponding points of an internal figure at another point (refer to \textcircled{2} of FIGS. 3(a) and 3(c)) is the widest. Furthermore, the interval between different points (refer to \textcircled{2} of FIGS. 3(b) and 3(d)) is wider than the interval between at least narrowest points (the first point). A form of the interval between points may be different depending on a form of a figure, but an interval at least first point is designated based on a corresponding eyesight value and is configured to be narrower than the interval between different points (hereinafter also referred to as a “second point”). Accordingly, the present invention is configured to check eyesight through user recognition for an interval at the first point. For example, an interval at the first point in a figure corresponding to an eyesight value 1.5 is designated so that it can be recognized by a testee who has resolution of the eyesight value 1.5. An interval at the first point corresponding to an eyesight value 0.1 is designated so that it can be visually recognized by a testee who has resolution of the eyesight value 0.1.

When the eyesight test chart image of FIG. 3 is displayed on the display unit 111, a response from a testee can be simplified. A form of an image between figures which is recognized by an eye of a testee is different because the size of a figure of an eyesight test chart image according to each eyesight value is different.

Eyesight is the ability to distinguish things. According to a classification criterion eyesight has the amount of rays recognized by an eye and resolution capable of recognizing two points that are spaced apart from each other. An eyesight chart is used to measure resolution that is the latter. A testee may recognize that the first point and a corresponding point have overlapped or recognize that the first point has been spaced apart from a corresponding point depending on his or her cognitive ability according to resolution. In the present invention, an eyesight test is performed using resolution between such points of a testee. More specifically, a direction does not need to be designated as in Landolt ring, and a testee has only to indicate whether points have simply overlapped (adhered to each other) or are spaced apart from each other. Furthermore, an eyesight test chart image according to an embodiment of the present invention provides a structure capable of further indicating the directions of the first point and a corresponding point. Accordingly, a simple user interface can be provided to an infant, a child and so on, and a separate eyesight chart does not need to be provided.

In a preferred example of eyesight test chart images of FIG. 3, the interval between the first point of the outer figure and a corresponding point of a corresponding inner figure and the interval between the second point of the outer figure and a corresponding point of a corresponding inner figure have been illustrated as being different in size, but is not limited thereto. The size of an interval at the first point and the size at an interval of the second point may be the same. At least, an interval (of the size) of the first point is set to check resolution of an eye. If eyesight test chart images are configured at the same interval, the automatic eyesight test device 100 can perform an eyesight test in response to a user response indicating whether only a single figure is seen or two figures are seen using only the button without a need to use the direction stick.

As described above, the display unit 111 displays an image including two figures expressed in a line.

Referring back to the blocks of FIG. 2, the lighting unit 113 output light to the display unit 111. The lighting unit 113 includes a plurality of white LEDs so that an image displayed on the display module of the display unit 111 can maintain constant brightness.

The optical unit 115 includes one or more lenses and a reflector so that an eye of a testee can recognize an image of the display unit 111. The optical unit 115 preferably includes two lenses. One of the lenses overlaps the other lens depending on eyesight test mode set by the control unit 117, so a testee can recognize an eyesight test chart image.

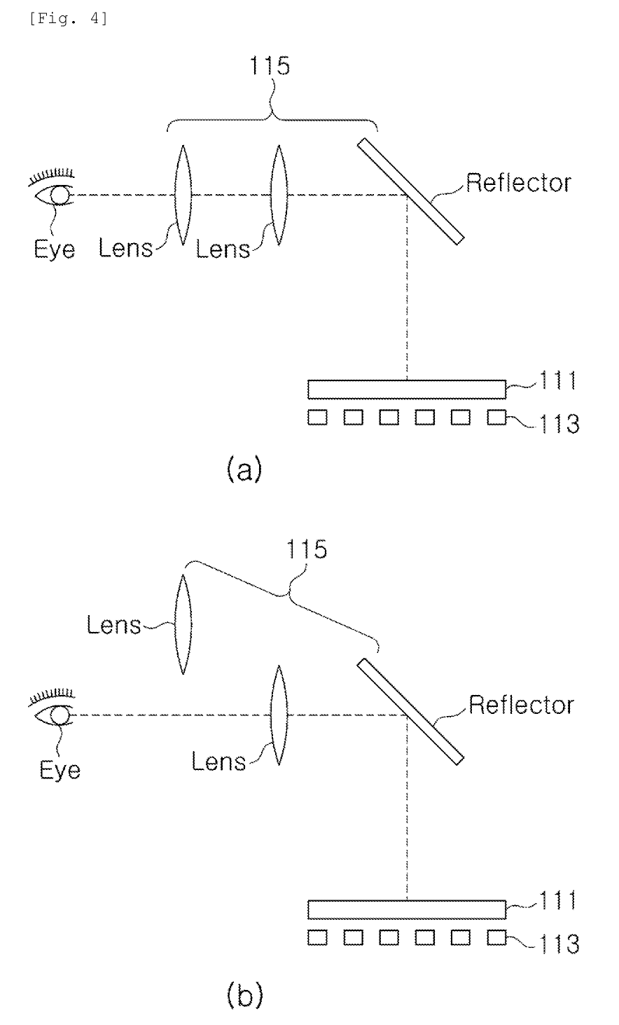

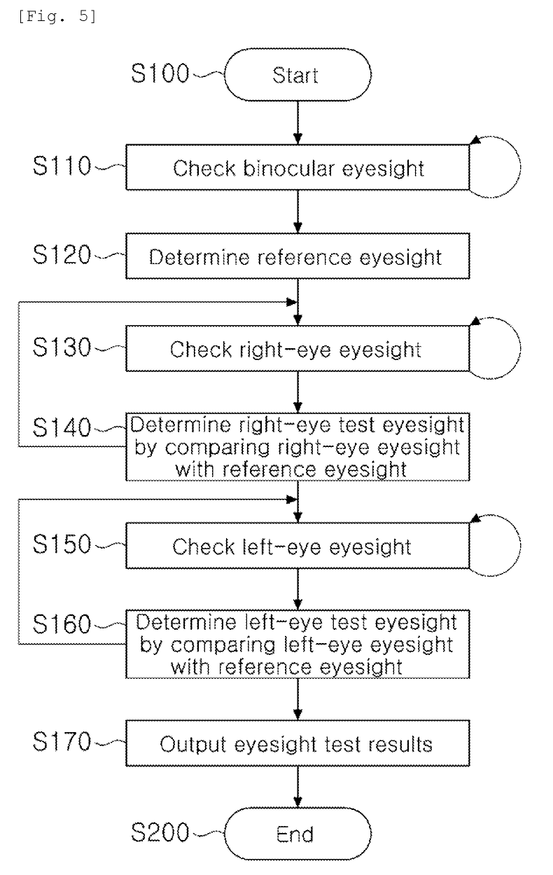

The optical unit 115 is described in more detail with reference to FIG. 4. FIG. 4 is a diagram showing an exemplary internal configuration of the automatic eyesight test device 100 for an eyesight test in long/short-distance mode.

FIG. 4(a) shows the structure of the optical unit 115 for an eyesight test in long-distance mode, and FIG. 4(b) shows the structure of the optical unit 115 for an eyesight test in short-distance mode.

As shown in FIG. 4(a), an eye recognizes light of an image displayed on the display unit 111 through two lenses and a reflector. The testee can recognize a long-distance eyesight test chart image through the overlap of the two lenses. Long-distance mode provides an eyesight test environment, such as that a testee sees an eyesight test chart at a distance of 5 m, for example.

FIG. 4(b) shows the structure of the optical unit 115 for an eyesight test in short-distance mode. Only one lens is located between an eye of a testee and the reflector, and the other lens is located at the place away from a light path along which an eye of a testee recognizes an eyesight test chart image through the optical unit 115 depending on the setting of the setting switch.

In FIG. 4(b), short-distance mode provides an eyesight test environment, such as that a testee sees an eyesight test chart at a distance of 50 cm, for example.

Referring back to the blocks of FIG. 2, the control unit 117 controls each of the blocks of the automatic eyesight test device 100. The control unit 117 controls other blocks by processing data according to a program using a program and data stored in the storage unit 103.

The control unit 117 responds to input from a testee or an administrator of the automatic eyesight test device 100 through the input unit 101, processes data in response to the input, and outputs corresponding results to the output unit 107. The control unit 117 includes an execution unit capable of executing code of a program, and may be a so-called processor, a microcomputer, CPU, MPU, etc.

The control unit 117 sets long-distance or short-distance mode in response to setting data received through the input unit 101, and controls the optical unit 115 based on the setting. For example, the control unit 117 may change the location of one of a plurality of lenses. Furthermore, the control unit 117 may perform a variety of types of control in response to control data received through the external interface unit 105, and may output corresponding results to the external interface unit 105. Furthermore, the control unit 117 may output an eyesight test guide comment in the form of a voice through the speaker of the output unit 107.

The control unit 117 recognizes a testee through the sensing unit 109, loads a program stored in the storage unit 103, and performs an eyesight test.

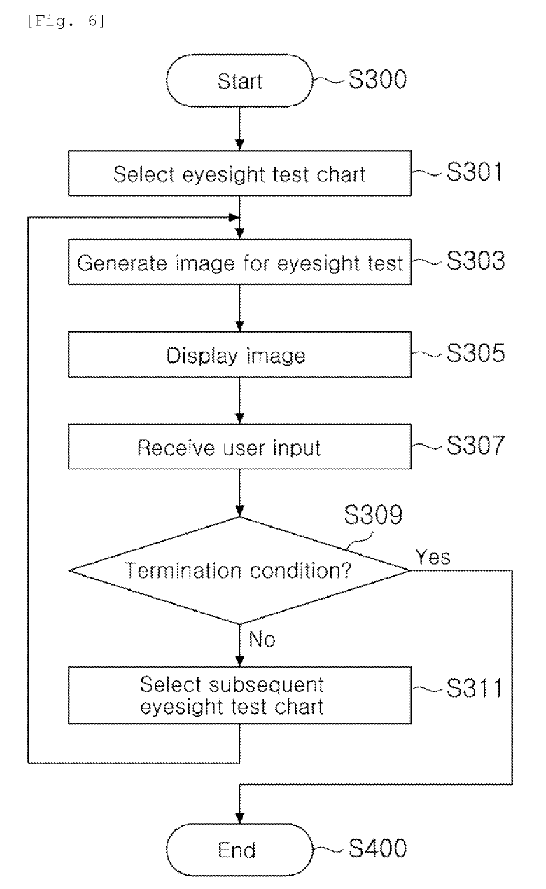

An eyesight test method executed based on the control unit 117 is described with reference to FIGS. 5 and 6.

The connection unit 119 enables data/control data to be transmitted and received between the blocks. The connection unit 119 includes a combination of one or more of a parallel bus, a serial bus and General Purpose Input Output (GPIO).

FIG. 5 is a diagram showing a flowchart for an eyesight test. The flowchart for an eyesight test in FIG. 5 is performed by the automatic eyesight test device 100, and is preferably performed by the control unit 117 by controlling other blocks using a program stored in the storage unit 103.

An eyesight test according to the present invention provides at least 3 types of test mode depending on a user environment. The first of the three test modes is a mode in which both a binocular and a monocular are checked (hereinafter referred to as a “first mode”), which may be used in a public place and an optician shop. The second of the three test modes is a monocular test mode (hereinafter referred to as a “second mode”), which may be used in an ophthalmic clinic. The third of the three test modes is a mode (hereinafter referred to as a “third mode”), which may be used in a medical examination, etc. Each of the first to third modes may include long-distance mode and short-distance mode.

The flowchart of FIG. 5 is described based on the second mode. The remaining modes are further described, if necessary.

First, an eyesight test is started in response to the recognition of a testee by the sensing unit 109 or user input from an administrator through the input unit 101 (S100).

Thereafter, the automatic eyesight test device 100 checks binocular eyesight in order to determine reference eyesight (S110). In order to check the binocular eyesight, the control unit 117 displays an eyesight test chart image on both the left-eye display module and the right-eye display module. The testee may input a response for the displayed image using the button or the direction stick of the input unit 101.

Step S110 may be performed several times. For example, step S110 is performed four times to determine four times of binocular eyesight. The steps of eyesight test charts used in the respective repetitions may be different. For example, in the first repetition, the 7-step eyesight test chart is used. After the second repetition, the 13-step eyesight test chart is used. Each of eyesight test chart images in the 7-step or 13-step eyesight test chart corresponds to a specific eyesight value. Accordingly, the 7-step eyesight test chart is used for a simple eyesight test, and the 13-step eyesight test chart is used for an accurate eyesight test. For a more accurate eyesight test, a subdivided eyesight test chart may be used.

As step S110 is repeated several times, at step S120, the automatic eyesight test device 100 determines reference eyesight. The automatic eyesight test device 100 may determine the same binocular eyesight of pieces of binocular eyesight determined by a plurality of binocular tests or binocular eyesight determined through a plurality of pieces of binocular eyesight or a probability to be the reference eyesight. The reference eyesight is eyesight checked for at least both eyes. The reference eyesight is subsequently used as a reference point in an eyesight test for a right eye or a left eye. The reference eyesight test has various purposes. For example, the reference eyesight test has an object of enabling a testee to be adapted to the automatic eyesight test device 100, and is subsequently used to filter eyesight test results in a right-eye eyesight test, a left-eye eyesight test and also a binocular eyesight test.

Thereafter, the automatic eyesight test device 100 displays an eyesight test chart image on the right-eye display module of the display unit 111 and checks right-eye eyesight (S130). In this case, only the right-eye display module of the display unit 111 is used, the eyesight test chart image is not displayed on the left-eye display module, and a corresponding LED of the lighting unit 113 that outputs light to the left-eye display module may not output light.

The right-eye eyesight test is performed using the 13-step eyesight test chart, for example, and is performed several times. For example, the right-eye eyesight test is performed twice. A single eyesight value for the right eye may be determined by the right-eye eyesight test.

The automatic eyesight test device 100 determines right-eye test eyesight by comparing the right-eye eyesight tested several times with the reference eyesight (S140). If the tested right-eye eyesight (values) falls within a constant critical range compared to the reference eyesight, it is recognized as reliable test results. The right-eye test eyesight may be determined based on a single piece of right-eye eyesight or an average of pieces of right-eye eyesight.

If the tested right-eye eyesight (values) does not falls within the constant critical range, the process returns to step 130 in which right-eye eyesight may be checked again.

Thereafter, the automatic eyesight test device 100 checks left-eye eyesight (S150). In this case, only the left-eye display module of the display unit 111 is used, and the eyesight test chart image is not display on the right-eye display module. Furthermore, a corresponding LED of the lighting unit 113 that outputs light to the right-eye display module may not output light.

The left-eye eyesight test is performed using the 13-step eyesight test chart, for example, and is performed several times. For example, the left-eye eyesight test is performed twice. A single eyesight value for the left eye may be determined by the left-eye eyesight test.

The automatic eyesight test device 100 determines left-eye test eyesight by comparing the left-eye eyesight tested several times with the reference eyesight (S160). If the tested left-eye eyesight (values) falls within a constant critical range compared to the reference eyesight, it is recognized as reliable test results. The left-eye test eyesight may be determined based on a single piece of left-eye eyesight or an average of pieces of left-eye eyesight.

If the tested left-eye eyesight (values) does not falls within the constant critical range, the process returns to step 150 in which left-eye eyesight may be checked again.

Thereafter, the automatic eyesight test device 100 output the eyesight test results through the output unit 107 (S170), transfers the eyesight test results to the external device through the external interface unit 105, and then terminates the eyesight test (S200).

In the first mode, binocular eyesight is checked several times after step S160, and binocular test eyesight may be further determined and output. In the third mode, a simple eyesight test chart may be used. For example, the eyesight test chart used at step S130 and step S150 may be the 7-step eyesight test chart.

The reference eyesight may be obtained through such a flow, and accurate eyesight of a testee can be checked using the reference eyesight.

The flowchart of FIG. 5 shows a flow of an eyesight test in long-distance mode or short-distance mode. In order to set mode as long-distance mode or short-distance mode, the control unit 117 may control the optical unit 115. For example, the control unit 117 may set long-distance mode or short-distance mode by controlling a motor (not shown) for changing the location of the lens or controlling a physical switch for changing the location, performs an eyesight test according to set mode in response to user input, and then output eyesight test results.

As described above, the control unit 117 performs an eyesight test of long-distance mode or short-distance mode in response to user input using the plurality of lenses of the optical unit 115, and output eyesight test results to the output unit 107.

FIG. 6 is a diagram showing a flowchart which uses the eyesight chart according to the present invention and is used to test binocular eyesight, left-eye eyesight or right-eye eyesight through simple user input. The flowchart of FIG. 6 is performed at least within step S110, step S130 or step S150 of FIG. 5, and is configured to respond to the display of an eyesight test chart image and corresponding user input. Accordingly, when step S110, step S130 or step S150 of FIG. 5 is performed, the flowchart of FIG. 6 is started (S300). The flowchart of FIG. 6 is also performed by the automatic eyesight test device 100 and is preferably performed using a program by the control unit 117. More specifically, the flowchart of FIG. 6 illustrates a flow in which an eyesight test is performed using user input operating in conjunction with an eyesight test chart image.

First, for a binocular eyesight, left-eye eyesight or right-eye eyesight test, the control unit 117 selects one eyesight chart data from the eyesight test chart of designated steps stored in the storage unit 103 (S301). The eyesight chart data includes an eyesight test chart image, for example, and further includes an eyesight value corresponding to the eyesight test chart image. Thereafter, the control unit 117 generates an image for an eyesight test using the eyesight chart data (S303). The generated image includes two figures expressed in a line.

In this case, the generation of the image means the generation of an image to be displayed on the display unit 111 from the eyesight chart data of the storage unit 103. For example, the generation of the image is performed by the copying of the eyesight test chart image of the storage unit 103 to a frame buffer or by scaling from the eyesight test chart image. The selected eyesight chart data is randomly selected or is the data of a middle step of the steps of the eyesight test chart or the eyesight chart data of an eyesight test chart corresponding to results (e.g., reference eyesight) determined in a previous eyesight test.

Thereafter, the control unit 117 displays an image, including the two figures expressed in a line, on the display unit 111 by outputting the image to the display unit 111 (S305). The control unit 117 may display the image for a left-eye eyesight test, right-eye eyesight test or binocular eyesight test to a testee by outputting the image to the left-eye display module and/or the right-eye display module.

The image displayed on the display module of the display unit 111 is recognized by the testee. The testee recognizes the image through one lens or a plurality of lenses. The displayed image displays an outer figure and an inner figure, displayed within the line of the outer figure, in a line form. The outer figure and the inner figure may be a ring type, a rectangle, a triangle or an ellipse or may have a specific another form. For simple image recognition, the outer figure and the inner figure may have the same form.

As may be seen from FIG. 3, the interval between the first point and a corresponding point in one displayed eyesight test chart image (e.g., FIG. 3(a), 3(b), 3(c) or 3(d)) has an interval of a specific size depending on an eyesight value, and preferably is different from the interval between the second point and a corresponding point and is the narrowest.

Thereafter, the testee inputs user input using the direction stick and/or the button. The control unit 117 receives user input that has responded to the displayed image through the input unit 101 (S307).

The user input is a response to the displayed image that is visually recognized by the testee (user) with respect to the first point of a line that expresses an outer figure and a corresponding point of the line of an inner figure.

For example, the user input is input for indicating whether figures have visually overlapped (adhered to each other) by a first point and a corresponding point using one button or two buttons or is input for indicating the direction of the first point and corresponding point from the center of an outer figure using the direction stick.

The eyesight test chart image displayed according to the present invention may be used to indicate whether points have overlapped and is used to indicate a direction. Accordingly, a simple input interface can be provided to the children and a finer eyesight test can be performed on the adult.

The control unit 117 determines a termination condition on the eyesight test (S309). The termination condition may be the number of times that image display (S305) is repeated, whether accurate eyesight test results according to user input have been obtained, etc.

If the termination condition is satisfied, the process returns to step S400 in which the control unit 117 terminates the flowchart of FIG. 6.

If the termination condition is not satisfied, the control unit 117 selects a subsequent eyesight chart from eyesight test charts stored in the storage unit 103 (S311). The subsequent eyesight chart data is determined by at least user input.

For example, when input indicative of overlap is received and/or an accurate direction is not indicated, the control unit 117 selects an eyesight chart having a smaller eyesight value (e.g., 1.0-->0.5) than corresponding eyesight chart data of an image displayed at step S305. Alternatively, when input indicative of non-overlap is received and/or an accurate direction is indicated, the control unit selects an eyesight chart having a greater eyesight value (e.g., 1.0-->1.5) than corresponding eyesight chart data of an image displayed at step S305.

The selection of the subsequent eyesight chart that has responded to the user input does not need to be sequentially with respect to an eyesight chart corresponding to each step of an eyesight test chart. For example, if an eyesight test chart includes eyesight values of 0.1, 0.3, 0.5, 0.7, 1.0, 1.5, 2.0, etc., 0.5 or 0.3 other than 0.7 may be selected as a subsequent eyesight chart in response to the reception of input indicative of the adherence for an eyesight test chart image of 1.0.

When the subsequent eyesight chart is selected, the control unit 117 repeatedly performs step S303 to step S309. Accordingly, the control unit 117 may display an image generated again in response to user input at step S305 that is repeatedly performed, and may receive user input again at step S307.

The image that is displayed again includes figures expressed in a line and is different from the image displayed at step S305 prior to the repeated execution. The size of the figures of the image that is displayed again may be set to be larger or smaller in response to user input.

Through the flow of FIG. 6, an eyesight test can be performed in response to simple user input using a simple image without using several images. Furthermore, an eyesight test can be automated because a subsequent image responding to input is generated. As described above, the automatic eyesight test device 100 and the automatic eyesight test method according to the present invention can solve various problems caused in the existing eyesight test method.

### INDUSTRIAL APPLICABILITY

The present invention described above may be substituted, modified and changed by a person having ordinary skill in the art to which the present invention pertains without departing from the technical spirit of the present invention, and thus is not restricted by the aforementioned embodiments and the accompanying drawings.

1. An automatic eyesight test method, comprising steps of:
(a) displaying an eyesight test chart image comprising a first figure expressed in a line and a second figure expressed in a line within the line of the first figure; and
(b) receiving user input which has responded to the displayed eyesight test chart image,
wherein an interval between a first point of the line expressing the first figure and a corresponding point of the line expressing the second figure is narrower than an interval between a second point of the line expressing the first figure and a corresponding point of the line expressing the second figure.

2. The automatic eyesight test method of claim 1, further comprising a step of (c) displaying an eyesight test chart image comprising a first figure expressed in a line and a second figure expressed in a line within the line of the first figure in response to the user input, wherein:
a size of the first figure and second figure included in the eyesight test chart image at the step (a) and a size of the first figure and second figure included in the eyesight test chart image at the step (c) are different, and
the size of the first figure and second figure at the step (c) is determined by user input.

3. The automatic eyesight test method of claim 2, wherein:
the eyesight test chart image at the step (a) corresponds to a first eyesight value, and
the eyesight test chart image at the step (c) corresponds to a second eyesight value different from the first eyesight value.

4. The automatic eyesight test method of claim 1, wherein the user input is a response to the eyesight test chart image visually recognized by a user with respect to the first point of the line expressing the first figure and the corresponding point of the line of the second figure.

5. The automatic eyesight test method of claim 4, wherein the user input is input indicating whether the first figure and the second figure have visually overlapped with respect to the first point and the corresponding point or input indicative of a direction recognized by the user for the first point and the corresponding point.

6. The automatic eyesight test method of claim 3, wherein:
the first figure and the second figure are figures of a ring type,
the step (a) comprises displaying only a single eyesight test chart image corresponding to the first eyesight value,
the step (c) comprises displaying only a single eyesight test chart image corresponding to the second eyesight value, and
the step (b) comprises receiving a response to a form of an eyesight test chart image visually recognized by a user with respect to a single eyesight test chart image corresponding to the first eyesight value as the user input.

7. An automatic eyesight test device, comprising:
a display unit for displaying an eyesight test chart image comprising a first figure expressed in a line and a second figure expressed in a line within the line of the first figure; and
an input unit for receiving user input which has responded to the displayed eyesight test chart image,
wherein an interval between a first point of the line expressing the first figure and a corresponding point of the line expressing the second figure is narrower than an interval between a second point of the line expressing the first figure and a corresponding point of the line expressing the second figure.

8. The automatic eyesight test device of claim 7, further comprising a control unit for generating the eyesight test chart image comprising a first figure and a second figure, wherein:
the control unit generates a subsequent eyesight test chart image which comprises a first figure expressed in a line and a second figure expressed in a line within the line of the first figure and differs from a previously generated eyesight test chart image in response to user input received through the input unit, and outputs the generated subsequent eyesight test chart image to the display unit,
a size of a first figure and second figure included in the previously generated eyesight test chart image and a size of the first figure and second figure included in the subsequent eyesight test chart image are different, and
the size of the first figure and second figure included in the subsequent eyesight test chart image is determined by the user input.

9. The automatic eyesight test device of claim 8, wherein:
the previously generated eyesight test chart image corresponds to a first eyesight value, and
the subsequent eyesight test chart image corresponds to a second eyesight value different from the first eyesight value.

10. The automatic eyesight test device of claim 8, further comprising:
an optical unit comprising a plurality of lenses; and
an output unit outputting eyesight test results,
wherein the control unit performs an eyesight test of long-distance mode or short-distance mode while operating in conjunction with user input using the plurality of lenses of the optical unit and outputs eyesight test results to the output unit.

11. The automatic eyesight test device of claim 9, wherein:
the first figure and the second figure are figures of a ring type,
the control unit outputs only the subsequent eyesight test chart image to the display unit in response to the user input, and
the user input is a response to a form of an eyesight test chart image visually recognized by a user with respect to a single eyesight test chart image corresponding to the first eyesight value.
